(19) United States
(12) Patent Application Publication
(10) Pub. No.: US 2010/0075897 A1
(43) Pub. Date: Mar. 25, 2010

(54) METHOD FOR SUSTAINEDLY RELEASING BIOACTIVE PEPTIDES AND APPLICATION THEREOF

(75) Inventors: Hongjian Li, Guangzhou (CN); Zhengding Su, Guangzhou (CN); Tianhong Zhou, Guangzhou (CN)

Correspondence Address: LAW OFFICES OF KHALILIAN SIRA, LLC, 9100 PERSIMMON TREE ROAD, POTOMAC, MD, 20854, US

(73) Assignee: JINAN UNIVERSITY

(21) Appl. No.: 12/236,343

(22) Filed: Sep. 23, 2008

(51) Int. Cl. A61K 38/16; A61P 35/00

(52) U.S. Cl. 514/12

(57) ABSTRACT

The present invention provides a method for sustainedly releasing bioactive peptide, comprising a bioactive peptide conjugated to a serum albumin binding peptide through a molecular linker so as to form a fusion polypeptide, in which the molecular linker is sensitive to plasma environment; and transferring the fusion polypeptide to a host, whereby plasma proteinase or alkaline pH of blood in the host can cleave the molecular linker to release the bioactive peptide therein. The fusion polypeptide is sensitive to plasma environment and the sustained release of bioactive peptide ensures the activity of released peptide, resulting in an increased circulation half-life in the host. The present invention also provides a method for using the fusion polypeptide drug as described above to treat human type 2 diabetes, human osteoporosis or cancer.

### BACKGROUND OF THE INVENTION

1. Field of the Invention

The present invention relates to biopharmaceutical application, particularly a method for sustainedly releasing bioactive peptides mediated by plasma proteinases or alkaline condition of blood.

2. Description of the Prior Arts

Peptide is short polymer formed from the linking, in a defined order, of α-amino acids which has no particular tertiary structure in aqueous solutions. Peptides with a small size of less than 6 KDa usually exert unique biological functions through specifically binding to protein receptors or adapters in cellular signal transduction pathways. Therefore, peptides or peptidomimetics are remedy for the urgent demanding for many diseases such as diabetes, cancer, AIDS etc, targeting aberrant protein-protein interactions, prior to effective small molecule drugs are obtained. However, the therapeutic potentials of these peptides is frequently limited by a short serum half-life, resulted from rapid enzymatic inactivation and clearance from the circulation. Accordingly, it is required to improve the pharmacokinetic properties of those peptides to enhance their efficacy in vivo.

Of many modern methods, it is a promising strategy to increase the circulation half-life of bioactive peptides by conjugating a bioactive peptide (tending to be removed after administration) with a plasma protein (natively occurring and having lower clearance rate) so as to form a single chain of fusion protein (Sheffield W. P., Cardiovacs. Haematol Disord. (2001), 1:1-5). Such fusion protein has clinical advantages of requiring less frequent injection and higher levels of the bioactive peptide in vivo. The aforementioned strategy resembles to the mechanism of some pathogens that evolved to recognize and attached to circulating proteins such as immunoglobulin, albumin, fibronectin or fibrinogen.

In practices, the pharmacokinetic properties of therapeutic proteins or peptides with short half-life are generally improved by binding to serum albumin. Serum albumin is the most abundant protein in the circulation system in mammals (40 g/L in human blood) Human serum albumin (HSA) is widely distributed throughout the human body, particularly in the intestinal and blood compartment, where it mainly relates to the maintenance of osmolarity. Human serum albumin (HSA) is a naturally occurring carrier that involves in endogenous transportation and is capable of delivering numerous natural and therapeutic molecules (Sellers et al., Albumin Structure, Function and Uses, Eds by Rosenoer V M. et al, Pergamon, Oxford, p159, 1977). One of its functions is to bind to molecules such as lipid and bilirubin. The half-life of HSA is 19 days (McCurdy T. R. et al., J. Lab. Clin. Med., (2004), 143:115-120), providing a promising method for prolonging the circulation half-life of therapeutic proteins or peptides. Several strategies have been reported to either covalently conjugate protein directly to serum albumins or to a protein or peptide that is capable of associating with the serum albumin, resulting in prolonged circulation half-life of proteins.

Albumin-binding peptides or Streptococcus G protein derivatives are used to prolong the half-life of proteins, which has been shown to have a rapid clearance in blood (U.S. Pat. No. 6,267,964). Roland Stork et al., report a strategy of improving the pharmacokinetic properties of antibody by fusing the antibody with an albumin binding domain (ABD) of Streptococcus G protein (Roland Stork, Dafine Müller and Roland E. Kontermann, Protein Engineering Design and selection, 2007 20: 569-576). The strategy is also used in developing a single-chain antibody (scDb, CEACD3), a fused antibody capable of targeting cytotoxic T lymphocytes to CEA-expressing tumor cells. The resulting trifunctional fusion protein (scDb-ABD) can be expressed in mammalian cells and recognizes two antigens including both human and mouse serum albumin. The strategy, which adds only a small protein domain (i.e., 46 amino acids) and which utilizes high affinity, non-covalent albumin interaction, should be promising to be widely used for improving the serum half-life of polypeptides.

Dafine Müller et al. construct several recombinant bispecific antibody-albumin fusion proteins and analyze their bioactivity and pharmacokinetic properties (Dafine Müller, Anette Karle, Bettina Meiβburger, Ines Höfig, Roland Stork and Roland E. Kontermann, J. Biol. Chem., (2007), 282: 12650-12660). These recombinant antibody formats were produced by fusing two different scFv molecules bispecific scDb or tafv molecules, respectively, to HSA. Those recombinant antibodies (scFv2-HSA, scDb-HSA and taFv-HSA) can retain their full binding ability and directly bind to tumor antigen, carcinoembryonic antigen and the T cell receptor, CD3, prior to fusion.

Dennis et al. employ relatively short peptides to bind serum albumin for fusing tumor-targeting bioactive compounds, which the peptides were selected from a phage display peptide library. (Mark S. Dennis, Min Zhang, Y. Gloria Meng, Miryam Kadkhodayan, Daniel Kirchhofer, Dan Combs and Lisa A. Damico, J. Biol. Chem., (2002), 277: 35035-35043). The half-lives of the fusions are longer than that of bioactive peptides alone and are similar to those of bioactive peptides covalently modified with PEG.

Moreover, serum albumin fusions provide novel and general methods for improving the pharmacokinetic properties of proteins which are rapidly cleared. Dennis et al. further conjugate albumin binding peptides (AB) to the antibody Fab4D5, i.e. monoclonal trastuzumab (HERCEPTIN®) to constitute a bifunctional molecule (AB-Fab4D5) capable of binding serum albumin and tumor antigen HER2 (erbB2) simultaneously (Mark S. Dennis, Hongkui Jin, Debra Dugger; Renhui Yang, Leanne McFarland, Annie Ogasawara, Simon Williams, Mary J. Cole, Sarajane Ross and Ralph Schwall, Cancer Res., (2007), 67: 254-61). More importantly, AB.Fab4D5 would not accumulate in kidney while Fab4D5 would, demonstrating that interaction of the bifunctional molecule with serum albumin alters the route of their clearance and metabolism. Rapid targeting, excellent tumor deposition and retention render AB.Fab a particular molecule for imaging and cancer therapy.

Vladimir Tolmachev et al. report that the excretion and absorption of targeted proteins in kidney can be effectively reduced via reversible binding to serum albumin (Vladimir Tolmachev, Anna Orlova et al., Cancer Res., (2007), 67: 2773-82).

As an alternative, therapeutic peptide or protein can be also directly fused to serum albumin. The conjugation of two extracellular immunoglobulin-like domains (V1, V2) of CD4 to HSA not only retain the original bioactivity of CD4, but also increases its half-life by 140-folds from 0.25±0.1 hours to 34 ±4 hours in an experimental rabbit model (Yeh et al., Proc. Natl. Acad. Sci. USA, (1992), 89: 1904-1910). Sung et al. observe that the half-life of interferon-β can be increased from 8 hours to 36 to 40 hours by being conjugated to human serum albumin (Sung et al, J. Interferon Cytokine Res., (2003), 23: 25-28).

Baggio et al report that a human glucagon-like peptide-1 (GLP-1)-albumin fusion protein (Albugon) has effects on stimulating the formation of GLP-1 receptor-dependent cAMP in BHK-GLP-1R cells (Baggio et al., Diabetes, (2004), 53: 2492-2500). However, compared with GLP-1 receptor agonist, exendin-4, the EC₅₀ of Albugon is reduced (0.2 vs. 20 mmol/mol). Jung-Guk Kim et al examine the in vivo bioactivity of the Albugon, which also names as CJC-1131 (Jung-Guk Kim et al., Diabetes, (2003), 52: 751-759). The experimental results demonstrate that Albugon mimics the action of native GLP-1, providing a new method for prolonged stimulation of GLP-1 receptor signaling. It is reported that association of insulin with serum albumin can slowly release insulin, providing an innovative concept for long-acting insulin (Yoram Shechter et al., Bioconjug. Chem., (2005), 16: 913-920). After subcutaneous injection of the long-acting insulin, the effect of lowering blood sugar delays for 0.5 to 1 hour and sustains for 12 hours.

The covalent linkage of peptide or protein drugs to human serum albumin or human serum albumin binding protein can greatly enhance their half-life in vivo, but is pharmaceutically irrelevant when it irreversibly inactivates them. Whilst these reports are said to have improved pharmacokinetic and pharmacodynamic properties, there is no disclosure or suggestion in these documentation of a remaining fully activity of peptides compared to the unconjugated bioactive peptides. There is no suggestion that further molecular design to release peptide from serum albumin-binding conjugate molecules or albumin binding peptide would be desirable.

To overcome these shortcomings, the present invention provides a method for sustainedly releasing bioactive peptide and application thereof to mitigate or obviate the aforementioned problems.

### SUMMARY OF THE INVENTION

The purpose of the present invention is to aim on the inadequacy of the conventional techniques and to provide a method for sustainedly releasing bioactive peptide, which is based on plasma proteinases or alkaline condition of blood to slowly release bioactive peptide from albumin-associated fusion protein or peptide. Released bioactive peptide remains its bioactive activity so as to extend its circulation half-life.

Another purpose of the present invention is to provide a method to prepare prolonged peptide drugs for clinical applications.

The rationale of the method in accordance with the present invention is to administrate a host a fusion polypeptide, comprising a bioactive peptide and a serum albumin binding peptide bridged with a molecular linker, which is cleavable by plasma proteases or alkaline pH condition of blood in the host. Once the fusion polypeptide is transferred into the host, plasma protease, or neutral or alkaline environment in the host as a switch can sustainedly release bioactive peptide embedded in the fusion polypeptide so as to achieve the goal of prolonging the half-life of bioactive peptide in circulation.

The fusion polypeptide of the method in accordance with the present invention is formed by linking bioactive peptide to serum albumin binding peptide (ABP) with a molecular linker sensitive to plasma environment, which comprises a structural formula of ABP-LK-PEP or PEP-LK-ABP.

In the structural formula as described above: ABP represents serum albumin binding peptide having an amino acid sequence of the following formula (I), that is a 12 mer amino acid sequence with high affinity to serum albumin:

(I)

Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂,

wherein

Xaa₁ is leucine;

Xaa₂ is proline;

Xaa₃ is any amino acid except cysteine;

Xaa₄ is any amino acid;

Xaa₅ is any amino acid;

Xaa₆ is a positively charged amino acid;

Xaa₇ is a hydrophobic amino acid;

Xaa₈ is a positively charged amino acid;

Xaa₉ is any amino acid except cysteine;

Xaa₁₀ is a hydrophobic amino acid;

Xaa₁₁ are proline; and

Xaa₁₂ is any amino acid.

In the amino acid sequence as shown in the formula I, the site and number of the conservative amino acid, leucine and proline in the ABP can be varied. When Xaa₁ is leucine, and Xaa₂ and Xaa₁₁ are proline, the serum albumin binding peptide in accordance with the present invention is Leu-Pro-Trp-His-Leu-Lys-Tyr-Arg-Glu-Pro-Pro-Arg or Leu-Pro-His-Ser-His-Arg-Ala-His-Ser-Leu-Pro-Pro.

The serum albumin binding peptides suitable for use in the present invention also include Ser-Leu-Phe-Arg-His-Gln-His-Ala-Thr-Pro-Gln-Ile, Ser-Leu-Leu-His-Trp-Thr-His-Lys-Ile-Pro-Ala-Leu, Lys-Tyr-Asn-His-Ser-His-Leu-Tyr-Trp-Gln-Arg-Pro, Asn-Val-Cys-Leu-Pro-Lys-Trp-Gly-Cys-Leu-Trp-Glu, Asp-Val-Cys-Leu-Pro-Gln-Trp-Trp-Gly-Cys-Lys-Trp-Gly, Asp-Ile-Cys-Leu-Pro-Arg-Trp-Gly-Cys-Leu-Trp-Glu or Asn-Ile-Cys-Leu-Pro-Arg-Trp-Gly-Cys-Leu-Trp-Asp and the like.

PEP represents one of bioactive peptides. When the peptides are fused to serum albumin binding peptides, their half-life can be enhanced. The peptides include human glucagon-like peptide-1 and the like, calcitonin, or peptides binding to Bcl-2 family apoptotic proteins (such as BH3 peptide of Bax protein) and the like.

LK represents a molecular linker capable of being cleaved by physiologically existing plasma proteases or mild alkaline condition of blood, such as thrombin recognition amino acid sequences or disulfide bonds. The molecular linker as described above serves as machinery to stepwise release bioactive peptide. If the molecular linker is thrombin recognition site, it includes an amino acid sequence of the following formula (II):

| | |
|---|---|
| Xaaⱼ-Xaaₖ-Xaaᵢ-Arg-Xaaₘ-Xaaₙ, | (II) |

wherein

Xaaⱼ is a hydrophobic amino acid or peptidyl bond;

Xaaₖ is a hydrophobic amino acid or peptidyl bond;

Xaaᵢ is proline or valine;

Xaaₘ is a non-acidic amino acid or peptidyl bond; and

Xaaₙ is a non-acidic amino acid or peptidyl bond.

The linker suitable for use in the present invention is Phc-Asn-Pro-Arg-Gly-Ala, Phe-Asn-Pro-Arg-Gly-Ser, Phe-Asn-Pro-Arg-Gly-Pro, Phe-Asn-Pro-Arg-Pro-Pro or Phe-Asn-Pro-Arg-Pro-Ala or the like.

When LK represents a disulfide bond, cyteines are introduced into the N-terminal of bioactive peptide and C-terminal of ABP respectively or vice versa, wherein the two peptides were linked by the disulfide bond.

Serum albumin binding peptides in accordance with the present invention are selected using M13 phage displayed peptide library, such as Ph.D-12™ phage library (New England Biolabs, Mass.), which is a linear randomized 12-mer peptide library with a diversity of 10⁹ and a titer of 10¹² pfu/mL.

The method in accordance with the present invention comprises following steps: immobilizing human serum albumin on a multi-well plate by physical absorption, allowing Ph.D.-12 library to interact with human serum albumin on multi-well plate, washing unbound phage, eluting and isolating bound phage, amplifying eluent and repeatedly screening, using universal primer-96gIII (for example, primer 5′-CCCTCATAGTTAGCGTAACG-3′ from New England Biolabs, Mass.) to perform the DNA sequencing analysis on phage particles eluted from the last three steps to deduce 12-mer peptide sequence with high affinity to human serum albumin, that is serum albunin binding peptide (ABP), including amino acid sequence of formula (I).

The serum albumin binding peptides (ABPs) in accordance with the present invention are obviously different from those found by Denis et al. (Denis et al. J. Biol. Chem. (2002), 277: 35035-35043). Peptide sequences found by Denis et al. are partially conservative and contain cysteines, whereas the conservative amino acids of 12-mer peptide sequence in accordance with the present invention do not include cysteine.

As used herein, the term “protein” or “polypeptide” refers to a polyaminoacid which can be either naturally occurring or synthetic, and has a molecular weight of large than 6 KDa. As used herein, the term “peptide” refers to a polyaminoacid which its molecular weight is less than 6 KDa. As used herein, the term “fusion polypeptide” refers to that two or more peptides are conjugated through peptide bonds. As used herein, the term “fusion protein” refers to that a protein conjugates with other proteins or peptides through peptide bonds.

Fusion polypeptide in accordance with the present invention can be prepared by standard solid-phase peptide synthesis technique, wherein the used peptide synthesizer is commercial product, for example, peptide synthesizer from Applied Biosystems, CA; reagents used for solid-phase peptide synthesis are available from chemical suppliers, for example, chemicals from NovaBiochem; the synthesis protocols are routinely employed by people in the art, including amino acid protection, coupling, decoupling or the like.

The fusion polypeptide in accordance with the present invention can be prepared by recombinant DNA technique which, for example, comprises step of: using pMFH/E. coli protein expression system (Su Z. D. et al., Protein Eng. Des. Sel., (2004), 17: 647-657) for fusing ABP-LK-PEP peptide gene sequence with a fusion protein carrier (such as MFH), expressing a fusion protein (such as MFH-ABP-LK-PEP), and releasing the fusion polypeptide through chemical cleavage. The affinity of the fusion polypeptide to human serum albumin is determined by surface plasmon resonance.

The present invention utilizes the physiological mild alkaline environment of blood as catalyst for reducing disulfide bonds to sustainedly release of bioactive peptide. At a condition of various pH value solutions, stability of disulfide bonds changes. Acidic environment can stabilize the disulfide bonds, while neutral or alkaline pH environment will reduce the disulfide bonds. In physiological condition, pH value of blood is slightly alkaline, for example, pH value of human blood stream is about 7.4, providing an excellent natural environment for spontaneously reducing disulfide bonds.

The present invention can also utilize physiologically existing biocatalyst to release bioactive peptide from fusion polypeptide. Plasma proteases in blood stream have the highest specificity and stringently regulated in physiological conditions. Thrombin is one of the most well-studied plasma proteases. There exists trace amount of active thrombin in normal people's blood, and the activity of thrombin in patient blood is slightly higher than those in the normal people (for example patients with diabetes and cancer). Hence, thrombin can function as a scissors to specifically cleave a molecular linker, i.e. LK which bridges serum albumin binding peptide (ABP) and bioactive peptide (PEP). Theoretically, the trace amount of thrombin is capable of acting on its subtract, i.e. a segment of amino acid sequence used in LK region, for the purpose of releasing peptide. The amino acid sequence, Leu-Val-Pro-Arg-Gly-Ser, is found to be the best subtract for the bovine thrombin and can also be recognized by human thrombin. However, comparing to bovine thrombin, human thrombin can more specifically recognize another amino acid sequence, that is Phe-Asn-Pro-Arg-Gly-Ser (Su Z. D. et al., Protein Eng. Des. Sel., (2004), 17: 647-657).

The method for sustainedly releasing bioactive peptide in accordance with the present invention can be utilized to prepare peptide drugs against human type 2 diabetes, human osteoporosis, cancer and other diseases, and the peptide drugs can be mixed with any pharmaceutically acceptable carriers.

Comparing to the conventional technique, the present invention has the following benefit effects:

1. The structure of the fusion protein used in the method for sustainedly releasing bioactive peptide according to the present invention ensures sustainedly physiological release of bioactive peptide, which not only prolongs the half-life of bioactive peptide in human circulation but also maintains physiological activity of the bioactive peptide;

2. The fusion polypeptide in accordance with the present invention has more effective pharmacokinetic properties than bioactive peptide alone; and

3. Although the fusion polypeptide used in the method for sustainedly releasing bioactive peptide in accordance with the present invention is formed by linking a peptide drug with a serum albumin binding peptide, by a molecular linker able to be cleaved by physiologically existing plasma protease or mild alkaline pH of blood, such that the whole fusion polypeptide is sensitive to plasma environment and the sustained release of bioactive peptide can be modulated according to the need of human body and the activity of released peptide can also be ensured.

Other objectives, advantages and novel features of the invention will become more apparent from the following detailed description when taken in combination with the accompanying drawings.

### BRIEF DESCRIPTION OF THE DRAWINGS

FIG. 1 shows curves of the binding ability of phage having peptides of SEQ ID NO. 1 to 9 to human serum albumin determined by ELISA;

FIG. 2 is high performance liquid chromatographic (HPLC) profile of a purified fusion polypeptide;

FIG. 3 is HPLC profile of a purified fusion polypeptide;

FIG. 4 shows surface plasmon resonance (SPR) sensorgrams of DP3.1 fusion polypeptide binding to human albumin;

FIG. 5 shows the peptide concentration of DP6.2 (i.e. the area under the peak) as a function of time during hydrolysis by human thrombin in the presence of HSA;

FIG. 6 shows the peptide concentration of DP3.1 as a function of time during hydrolysis by both human thrombin and DPP IV in the presence of HSA; and

FIG. 7 shows the peptide concentration of DP4.1 as a function of time during hydrolysis by human thrombin and DPP IV in the presence of HSA.

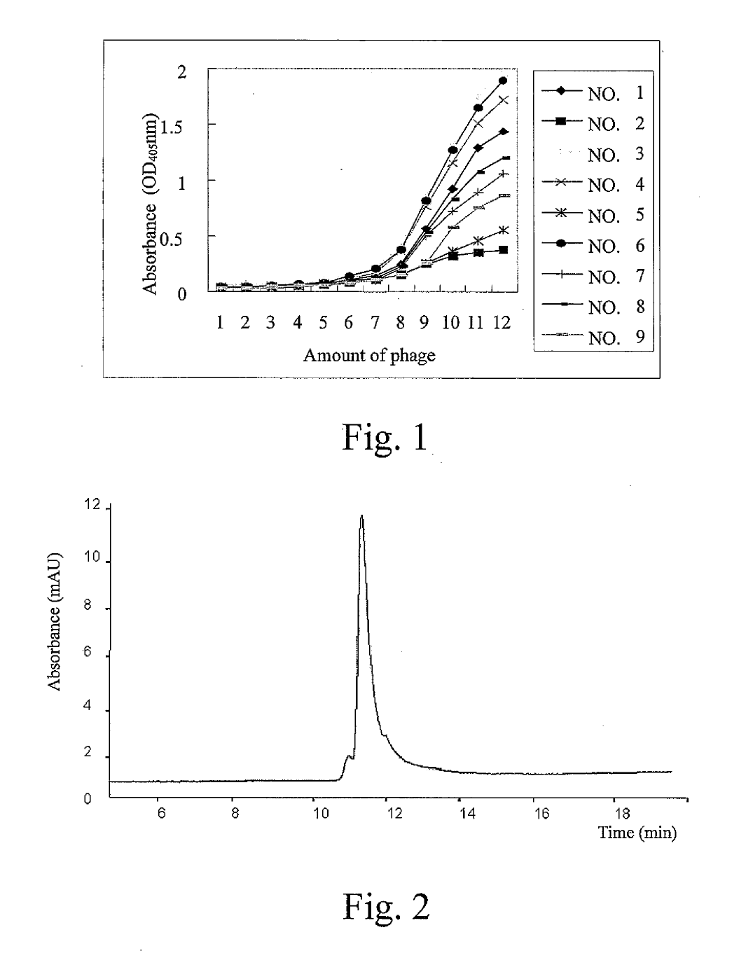

### DETAILED DESCRIPTION OF THE PREFERRED EMBODIMENTS

### Example 1

### Selection of Human Serum Albumin Binding Peptides by Phage Display

The present example employed Ph.D-12™ phage display peptide library from New England Biolabs, Inc. for selecting human serum albumin binding peptides. Ph.D-12™ phage display peptide library was designed based on M13 phage, comprising randomized linear 12-mer peptides. The library has a diversity of 10⁹ and its titer is 10¹² pfu/mL.

Selecting human serum albumin binding peptide was performed by four runs of panning. Human albumin (Sigma-Aldrich, St-Louis, USA) was dissolved in 0.1 mol/L NaHCO₃ buffer (pH 8.6) to prepare a solution containing 100 μg/mL human albumin (pH 8.6). The four runs of screening were listed as following:

The First-Run Screening:

1. Adding 1.5 mL human serum albumin solution (100 μg/mL, pH 8.6) to a sterilized polystyrene plate (60×15 mm), followed by placing the plate in a humidity incubator at 4° C. and gently agitating overnight (This step is to immobilize human serum albumin on the surface of plate. This step is also important for eliminating phage binding to the surface of plate);

2. Dissolving phage library at an amount of 10 μL in 1 mL TBS buffer containing 0.1% (v/v) Tween-20 and adding the diluted phage library onto the uncoated plate and incubating the prepared phage library with plate by gently agitating to remove any phage with non-selective to the surface of plate wells; and unbound phage library was used for biopanning peptides specific for human serum albumin;

3. Adding the prefiltered phage library onto the coated plate and incubating the prepared phage library with human serum albumin by gently agitating to allow phage to bind to human serum albumin;

4. After incubation, repeatedly washing with TBS buffer containing 0.1% (v/v) Tween-20 to remove unbound phage;

5. Dissolving human serum albumin in 2 mol/L Glycine-HCL (pH2.2) to prepare a solution at a concentration of 1 mg/mL, using the solution to elute the bound phage, wherein time of the elution was less than 10 minutes, and then immediately adding 150 μL of 1 mol/L Tris-HCl (pH9.1) to neutralize the eluted phage; and

6. Obtaining a few amount of eluted phage to determine the concentration of phage with E. coli. ER2738 by titration and amplifying the rest of eluted phage: diluting overnight culture of E. coli ER2738 to a ratio of 1:100 with LB medium; adding 1 mL of the diluted culture into tube; picking a blue plaque from plate having less than 100 phage plagues with sterilized toothpick and transferring to the tube containing the diluted culture, agitating at 37° C. for 5 hours, followed by transferring the culture to a microcentrifuge tube, And centrifuging at 12,000 for 10 minutes, collecting supernatant as amplified phage library; aspiring 80% supernatant and stored at 4° C., generally titration thereof would be maintained within a few weeks.

The Second-Run of Screening:

Adding 1.5 mL of 100 μg/mL human serum albumin (pH 8.6) to a sterilized polystyrene plate (60×15 mm), followed by placing the plate in a humidity incubator at 4° C. and gently agitating overnight, adding the amplified phage library obtained from the first-run of screening to perform the second run screening as described in the first run.

The Third-Run of Screening:

Adding 1.5 mL of 100 μg/mL human serum albumin (pH8.6) to a sterilized polystyrene plate (60×15 mm), followed by placing the plate in a humidity incubator at 4° C. and gently agitating overnight, adding the amplified phage library obtained from the second-run of screening to perform the third run screening as described in the first run.

The Fourth-Run of Screening:

Adding 1.5 mL of 100 μg/mL human serum albumin (pH 8.6) to a sterilized polystyrene plate (60×15 mm), followed by placing the plate in a humidity incubator at 4° C., gently agitating overnight, adding the amplified phage library obtained from the third-run of screening, adding the prepared phage library to the coated plate and incubating phage library with human serum albumin by gently agitating to allow phages binding to human serum albumin for 20 minutes, washing to remove unbound phage with TBS buffer containing 0.3% (v/v) Tween-20. The fourth-run of screening is the same as the first-run of screening except for determining titer of and amplifying the phage.

Randomly selecting 10 to 20 clones of phage eluted from the second- and third-run of screenings to be subject to DNA sequencing, employing universal primer 96 gIII (with sequence of 5′-CCC TCA TAG TTA GCG TAA CG-3′). Table 1 summarized amino acid sequence deduced from the results of DNA sequencing.

TABLE 1

The amino acid sequences of peptides with high
affinity to human serum albumin being selected
from phage display library

| | |
|---|---|
| SEQ ID NO. | Amino acid sequence |

| | |
|---|---|
| 1 | NVCLPKWGCLWE |
| 2 | DVCLPQWGCLWG |
| 3 | DICLPRWGCLWE |
| 4 | NICLPRWGCLWD |
| 5 | LPWHLKYREPPR |
| 6 | LPHSHRAHSLPP |
| 7 | SLFRHQHATPQI |
| 8 | SLLHWTHKIPAL |
| 9 | KYNHSHLYWQRP |

### Example 2

### Determining Affinity of the Selected Phage to Human Serum Albumin by Enzyme-Linked Immunosorbent Assay (ELISA)

Selected individual phage clones respectively comprising peptides of SEQ ID NO. 1 to 9 as indicated in Table 1 were numbered as phage ID NO. 1 to 9 as shown in Table 2. The selected phage clones were used for the present example. Individual phage were amplified and purified, and used for characterizing their binding affinities with human serum albumin.

Human serum albumin was diluted with 0.1 mol/L NaHCO₃ to obtain a solution at a concentration of 100 μg/mL. The obtained solution was loaded to each row of wells of ELISA multiple-well plate. To each well of the plate was added 200 μL the solution. The plate was then placed in a humidity environment at 4° C. overnight. Another multiple-well plate was used for serial dilution of phage. The two plates were loaded with 1% casein solution (in 0.1 mol/L NaHCO₃). Each group of phage was subject to four-fold serial dilution to allow about 10¹² phage particles in the first well and about 2.0×10⁵ phage particles in the last well. Each row of the diluted phage was transferred into the plate coated with human albumin by using multi-channel micropipette. The plate was agitated at room temperature for 1 hour, repeatedly washed with TBS buffer containing 0.3% (v/v) Tween-20, followed by incubation with rabbit anti-M13 phage antibody, and then incubation with goat anti-rabbit IgG conjugated with horseradish peroxidase (HRP) for determining bound phage.

The amount of bound horseradish peroxidase was determined at 405 nm by adding ABTS/H₂O₂ substrates. Each sample was examined in triplicate. Table 2 summarized the absorbance of each group determined at 405 nm. In control group, without adding phage, Table 3 compiled statistics of background absorbance of subtract under 405 nm. The absorbance of bound phage at 405 nm by ELISA was proportional to the amount of bound phage.

TABLE 2
Affinity of bound phage to albumin determined by ELISA

| Phage | | | | | | | | | | | | | |
|---|---|---|---|---|---|---|---|---|---|---|---|---|---|
| ID NO. | | 1 | 2 | 3 | 4 | 5 | 6 | 7 | 8 | 9 | 10 | 11 | 12 |
| 1 | Sample | 0.084 | 0.090 | 0.093 | 0.108 | 0.126 | 0.156 | 0.183 | 0.291 | 0.610 | 0.976 | 1.344 | 1.492 |
| 1 | Blank | 0.040 | 0.044 | 0.044 | 0.044 | 0.045 | 0.048 | 0.049 | 0.050 | 0.050 | 0.051 | 0.051 | 0.058 |
| 2 | Sample | 0.068 | 0.071 | 0.075 | 0.084 | 0.098 | 0.115 | 0.141 | 0.178 | 0.279 | 0.356 | 0.389 | 0.411 |
| 2 | Blank | 0.035 | 0.035 | 0.035 | 0.035 | 0.036 | 0.036 | 0.035 | 0.035 | 0.035 | 0.036 | 0.038 | 0.039 |
| 3 | Sample | 0.095 | 0.097 | 0.100 | 0.117 | 0.140 | 0.187 | 0.260 | 0.461 | 0.901 | 1.402 | 1.831 | 2.036 |
| 3 | Blank | 0.037 | 0.038 | 0.041 | 0.041 | 0.043 | 0.045 | 0.046 | 0.053 | 0.055 | 0.055 | 0.061 | 0.063 |
| 4 | Sample | 0.080 | 0.081 | 0.094 | 0.095 | 0.115 | 0.163 | 0.214 | 0.409 | 0.811 | 1.208 | 1.571 | 1.781 |
| 4 | Blank | 0.041 | 0.041 | 0.043 | 0.043 | 0.044 | 0.047 | 0.048 | 0.048 | 0.053 | 0.055 | 0.055 | 0.058 |
| 5 | Sample | 0.071 | 0.076 | 0.077 | 0.086 | 0.104 | 0.123 | 0.155 | 0.198 | 0.302 | 0.406 | 0.501 | 0.593 |
| 5 | Blank | 0.040 | 0.040 | 0.041 | 0.041 | 0.043 | 0.043 | 0.045 | 0.045 | 0.046 | 0.047 | 0.047 | 0.048 |
| 6 | Sample | 0.085 | 0.090 | 0.094 | 0.110 | 0.125 | 0.181 | 0.253 | 0.431 | 0.883 | 1.333 | 1.723 | 1.969 |
| 6 | Blank | 0.043 | 0.043 | 0.044 | 0.046 | 0.046 | 0.047 | 0.055 | 0.061 | 0.064 | 0.067 | 0.069 | 0.077 |
| 7 | Sample | 0.080 | 0.080 | 0.081 | 0.091 | 0.110 | 0.129 | 0.143 | 0.251 | 0.543 | 0.761 | 0.931 | 1.105 |
| 7 | Blank | 0.043 | 0.043 | 0.044 | 0.044 | 0.044 | 0.044 | 0.043 | 0.044 | 0.047 | 0.044 | 0.047 | 0.048 |
| 8 | Sample | 0.078 | 0.082 | 0.085 | 0.101 | 0.121 | 0.141 | 0.164 | 0.267 | 0.575 | 0.875 | 1.114 | 1.250 |
| 8 | Blank | 0.040 | 0.040 | 0.042 | 0.043 | 0.043 | 0.044 | 0.044 | 0.046 | 0.048 | 0.049 | 0.049 | 0.049 |
| 9 | Sample | 0.077 | 0.078 | 0.085 | 0.097 | 0.097 | 0.120 | 0.149 | 0.193 | 0.299 | 0.619 | 0.804 | 0.910 |
| 9 | Blank | 0.043 | 0.043 | 0.043 | 0.044 | 0.044 | 0.044 | 0.045 | 0.047 | 0.048 | 0.049 | 0.049 | 0.051 |

The sample numbers in Table 2 as well as Table 3 (see below), i.e. Phage ID NO. 1 to 9 represented nine selected phage samples, which express nine different peptides, as described in Table 1, respectively. For example, the Phage ID NO. 1 in Table 2 represented phage containing peptide with amino acid sequence of NVCLPKWGCLWE corresponding to the SEQ ID NO. 1 in Table 1.

TABLE 3
Absorbance of 9 selected phage determined by ELISA

| Phage | | | | | | | | | | | | |
|---|---|---|---|---|---|---|---|---|---|---|---|---|
| ID NO. | 1 | 2 | 3 | 4 | 5 | 6 | 7 | 8 | 9 | 10 | 11 | 12 |
| 1 | 0.040 | 0.046 | 0.049 | 0.064 | 0.081 | 0.108 | 0.134 | 0.241 | 0.560 | 0.925 | 1.293 | 1.434 |
| 2 | 0.033 | 0.036 | 0.040 | 0.049 | 0.062 | 0.079 | 0.106 | 0.143 | 0.244 | 0.320 | 0.351 | 0.372 |
| 3 | 0.058 | 0.059 | 0.059 | 0.076 | 0.097 | 0.142 | 0.214 | 0.408 | 0.846 | 1.347 | 1.770 | 1.973 |
| 4 | 0.039 | 0.040 | 0.051 | 0.052 | 0.071 | 0.116 | 0.166 | 0.361 | 0.758 | 1.153 | 1.516 | 1.723 |
| 5 | 0.031 | 0.036 | 0.036 | 0.045 | 0.061 | 0.080 | 0.110 | 0.153 | 0.256 | 0.359 | 0.454 | 0.545 |
| 6 | 0.042 | 0.047 | 0.050 | 0.064 | 0.079 | 0.134 | 0.198 | 0.370 | 0.819 | 1.266 | 1.654 | 1.892 |
| 7 | 0.037 | 0.037 | 0.037 | 0.047 | 0.066 | 0.085 | 0.100 | 0.207 | 0.496 | 0.717 | 0.884 | 1.057 |
| 8 | 0.038 | 0.042 | 0.043 | 0.058 | 0.078 | 0.097 | 0.120 | 0.221 | 0.527 | 0.826 | 1.065 | 1.201 |
| 9 | 0.034 | 0.035 | 0.042 | 0.053 | 0.053 | 0.076 | 0.104 | 0.146 | 0.251 | 0.570 | 0.755 | 0.859 |

Phage ID No. 1 to 9 represented nine different phage containing nine individual peptides as described in Table 1. For example, Phage ID No. 1 herein represented phage containing peptide with an amino acid sequence of NVCLPKWGCLWE corresponding to the SEQ ID NO. 1 in Table 1.

### Example 3

### Preparing ABP-LK-GLP Fusion Polypeptide Against Type 2 Diabetes by Recombinant DNA Technology

In present example, human glucagon-like peptide-1 (GLP-1) was taken as an example for design of a novel therapeutic peptide to treat type 2 diabetes.

About 90% diabetes belongs to type 2 diabetes. Type 2 diabetes was also known as non-insulin dependent diabetus mellitus (NIDDM). Patients with type 2 diabetes generally could produce insulin, but the produced insulin could not be utilized by cells in the body.

Human glucagon like peptide-1 (GLP-1) has been proved to be effective in lowering the concentration of blood sugar since it was found in 1984. More importantly, there was almost no risk in lowering blood sugar when using GLP-1. Since human GLP-1 only stimulated secretion of native glucose-induced insulin. GLP-1 could induce lots of biological effects, for example, stimulating secretion of insulin, inhibiting secretion of glucagon, inhibiting gastric emptying, elevating uptake of glucose and inducing weight loss. In addition, pre-clinical research showed GLP-1 was able to prevent β-cell regression during progression of diabetes. Its most outstanding characters might lie in that it could stimulate insulin secretion without causing risk of over-lowering blood sugar, in contrast to insulin therapy or some oral medicine which would induce insulin expression usually caused risk of over-lowering blood sugar.

Human glucagon like peptide-1 had its distinct and beneficial effects on type 2 diabetes to become a potential drug. Maintaining physical level of human GLP-1 via gene therapy could alleviate hyperglycemia and maintain blood sugar level normal in a long term. Recently, research on selected small-molecule agonist for human GLP-1 receptor was set forth. There would be lots of work prior to clinical use. Collectively, methods for treating type 2 diabetes with polypeptide were greatly accepted by people, because the interaction between human GLP-1 and its receptor involved in a large interface. In the last decade, various human GLP-1 analogues were designed out by mutating and substituting side-chain of human GLP-1, however, except for Exendin-4, there was no prominent progress in the field. Moreover, these human GLP-1 analogues either lost their activity or were cleared rapidly.

Human GLP-1 was an endogenous polypeptide including 30 or 31 amino acids, generated by cleavage of glucagon precursor and comprised two native structures, GLP-1 (7-36) amide and GLP-1 (7-37), wherein the amino acid sequence of GLP-1 (7-36) amide is HAEGTFTSDVSSYLEGQAAKEFIAWLVKGR-NH₂; and the amino acid sequence of GLP-1 (7-37) is HAEGTFTSDVSSYLEGQAAKEFIAWLVKGRG.

Human GLP-1 as a therapeutic drug was limited by its rapid degradation by dipeptidase, DPP IV, and neutral endopeptidase, NEP24.11. N-terminal of human GLP-1 interacted with the core domain of the receptor and C-terminal of human GLP-1 assure its selectivity via interacting with N-terminal of the receptor. Therefore, the challenge for improving that drug is at constructing a stable human GLP-1 analogue having a long half-life. Table 4 listed a serial of fusion polypeptides of sustained-release human glucagon-like peptide-1 (GLP-1) designed according to ABP-LK-GLP fusion polypeptide model. ABP-LK-GLP fusion polypeptide was linked to MFH fusion carrier via methionine (Met or M) or aspartate-proline (Asp-Pro or DP) to form a fusion protein, wherein the linkage was prone to chemical cleavage. The ABP-LK-GLP fusion polypeptides were prepared by recombinant DNA methods (Osborne J. M. and Su Z. D. et al., J. Biomol. NMR, (2003), 26: 317-326; Su Z. D. et al, Protein Eng. Des. Sel., (2004), 17:647-657; Li H. J. et al., Protein Expression & Purification, (2006), 50:238-46; Su Z. D. et al., U.S. Pat. No. 7,390,63). After chemical cleavage, methionine (i.e. M or Met) bridging between MFH fusion carrier and ABP-LK-GLP fusion polypeptide was removed from ABP-LK-GLP fusion polypeptide together with MFH fusion carrier. Aspartate (i.e. D or Asp) in aspartate-proline (i.e. DP or Asp-Pro) bridging between WFH fusion carrier and ABP-LK-GLP fusion polypeptide was removed from ABP-LK-GLP fusion polypeptide together with MFH fusion carrier, while proline (i.e. P or Pro) was left at N-terminal of ABP-LK-GLP fusion polypeptide. Analysis demonstrated that proline left on ABP-LK-GLP did not affect the affinity of ABP-LK-GLP to serum albumin.

TABLE 4

Sustained release human glucagon-like
peptide-1 analogues

| | |
|---|---|
| Amino acid sequence | Name |

| | |
|---|---|
| PDICLPRWGCLWEFNPRGA HAEGTFTSDVSSYLEGQAAKEFIA | DP3.1 |
| WLVKGRG | |
| PDICLPRWGCLWEFNPRGP HAEGTFTSDVSSYLEGQAAKEFIA | DP3.2 |
| WLVKGRG | |
| PDICLPRWGCLWEFNPRGS HAEGTFTSDVSSYLEGQAAKEFIA | DP3.3 |
| WLVKGRG | |
| PNICLPRWGCLWDFNPRGA HAEGTFTSDVSSYLEGQAAKEFIA | DP4.1 |
| WLVKGRG | |
| PNICLPRWGCLWDFNPRGP HAEGTFTSDVSSYLEGQAAKEFIA | DP4.2 |
| WLVKGRG | |
| PNICLPRWGCLWDFNPRGS HAEGTFTSDVSSYLEGQAAKEFIA | DP4.3 |
| WLVKGRG | |
| PLPHSHRAHSLPPFNPRGA HAEGTFTSDVSSYLEGQAAKEFIA | DP6.1 |
| WLVKGRG | |
| PLPHSHRAHSLPPFNPRGP HAEGTFTSDVSSYLEGQAAKEFIA | DP6.2 |
| WLVKGRG | |
| PLPHSHRAHSLPPFNPRGS HAEGTFTSDVSSYLEGQAAKEFIA | DP6.3 |
| WLVKGRG | |
| PSLLHWTHKIPALFNPRGA HAEGTFTSDVSSYLEGQAAKEFIA | DP8.1 |
| WLVKGRG | |
| PSLLHWTHKIPALFNPRGP HAEGTFTSDVSSYLEGQAAKEFIA | DP8.2 |
| WLVKGRG | |
| PSSLLHWTHKIPALFNPRGS HAEGTFTSDVSSYLEGQAAKEFI | DP8.3 |
| A WLVKGRG | |
| DICLPRWGCLWEFNPRGA HAEGTFTSDVSSYLEGQAAKEFIA | M3.1 |
| WLVKGRG | |
| DICLPRWGCLWEFNPRGP HAEGTFTSDVSSYLEGQAAKEFIA | M3.2 |
| WLVKGRG | |
| DICLPRWGCLWEFNPRGS HAEGTFTSDVSSYLEGQAAKEFIA | M3.3 |
| WLVKGRG | |
| NICLPRWGCLWDFNPRGA HAEGTFTSDVSSYLEGQAAKEFIA | M4.1 |
| WLVKGRG | |
| NICLPRWGCLWDFNPRGP HAEGTFTSDVSSYLEGQAAKEFIA | M4.2 |
| WLVKGRG | |
| NICLPRWGCLWDFNPRGS HAEGTFTSDVSSYLEGQAAKEFIA | M4.3 |
| WLVKGRG | |
| LPHSHRAHSLPPFNPRGA HAEGTFTSDVSSYLEGQAAKEFIA | M6.1 |
| WLVKGRG | |
| LPHSHRAHSLPPFNPRGP HAEGTFTSDVSSYLEGQAAKEFIA | M6.2 |
| WLVKGRG | |
| LPHSHRAHSLPPFNPRGS HAEGTFTSDVSSYLEGQAAKEFIA | M6.3 |
| WLVKGRG | |
| SLLHWTHKIPALFNPRGA HAEGTFTSDVSSYLEGQAAKEFIA | M8.1 |
| WLVKGRG | |
| SLLHWTHKIPALFNPRGP HAEGTFTSDVSSYLEGQAAKEFIA | M8.2 |
| WLVKGRG | |
| SLLHWTHKIPALFNPRGS HAEGTFTSDVSSYLEGQAAKEFIA | M8.3 |
| WLVKGRG | |
| LPHSHRAHSLPPFNPRPP HAEGTFTSDVSSYLEGQAAKEFIA | PP6.2 |
| WLVKGRG | |
| LPHSHRAHSLPPFNPRPA HAEGTFTSDVSSYLEGQAAKEFIA | PA6.2 |
| WLVKGRG | |

GLP-1 (7-37) gene was amplified by a standard PCR method by using two partially overlapping 5′-oligonucleotide and 3′-oligonucleotide as primers and GLP-1 gene in plasmid pCMFH-GLP-1 (Li H. J. et al., Protein Expression & Purification, (2006), 50: 238-46) as template. Primers were designed based on E. Coli codon preference. 5′-oligonucleotide primer was for introducing the DNA sequence of ABP and LK at 5′-end of GLP-1 (7-37) gene. Meanwhile 5′-end and 3′-end of PCR products were introduced with EcoRI and BamHI restriction enzyme sites respectively. The resulting PCR products were DNA fragments encoding recombinant polypeptide ABP-LK-GLP. The PCR products were purified with Qiagen PCR product purification kit (Mississauga, ON), digested with EcoRI and BamHI to form sticky ends. The enzyme-digested DNA fragments were then ligated with pMFH-MCS expression vector treated with the same restriction enzymes individually. The constructed expression plasmids of recombinant ABP-LK-GLP-1 fusion polypeptides were confirmed by DNA sequencing and respectively named as pMFH-DP3.1, pMFH-DP3.2, pMFH-DP3.3, pMFH-DP4.1, pMFH-DP4.2, pMFH-DP4.3, pMFH-DP6.1, pMFH-DP6.2, pMFH-DP6.3, pMFH-DP8.1, pMFH-DP8.2, pMFH-DP8.3, pMFH-M3.1, pMFH-M3.2, pMFH-M3.3, pMFH-M4.1, pMFH-M4.2, pMFH-M4.3, pMFH-M6.1, pMFH-M6.2, pMFH-M6.3, pMFH-M8.1, pMFH-M8.2, pMFH-M8.3, pMFH-PP6.2 and pMFH-PA6.2.

The plasmids as described above were transformed into E. coli BL21 (DE3) for expressing fusion proteins.

Purification of the fusion proteins was performed with Ni-NTA agarose resins by a standard protocol, comprising steps as following:

(1) Inoculating the transformed E. coli into 50 mL LB medium containing 100 μg/mL ampicillin, culturing overnight, transferring to 1 L LB medium containing the same concentration of ampicillin and culturing at 37° C.;

(2) While OD₆₀₀ₙₘ of the culture reached 0.8, adding IPTG at 1 mmol/L for induction and culturing at 37° C. for 12 hours and then centrifuging at 6000 rpm for 20 minutes to collect cells;

(3) Resuspending cell pellet with a buffer containing 6 mol/L urea, 20 mmol/L Tris-HCl (pH8.0) and 100 mmol/L NaCl, and gently agitating for minutes;

(4) Ultrasonicating the cells for 1 minute and centrifuging at 10,000 rpm for 30 minutes to collect the supernatant; and

(5) Equilibrating Ni-NTA resins column with lysis buffer (50 mmol/L Tris pH8.0), 100 mmol/L NaCl and 6 mol/L urea), loading the above supernatant after equilibrium, followed by using lysis buffer containing mmol/L, 20 mmol/L, 30 mmol/L and 40 mmol/L imidazole for washing, eluting with lysis buffer containing 200 mmol/L imidazole from Ni-NTA resin column to obtain an elution containing target proteins, desalting the elution by use of C18 Sep-Pak column and lyophilizing the desalted elution to powder.

The purified fusion protein was verified by SDS-PAGE, HPLC and mass spectrometry, as shown in FIG. 2, the purity of purified fusion protein was over 99%. Protein concentration was calculated by absorbance at OD₂₈₀ₙₘ (Gill and von Hipple, Anal. Biochem., (1989), 182: 319-26).

The purified fusion protein was hydrolyzed with 70% formic acid (with or without cyanobromide) to release fusion polypeptide ABP-LK-GLP. If the 70% formic acid with cyanobromide was used, crystalline cyanobromide (CNBr) was added (in a mole ratio of 100:1=CNBr:Met residue) and stand at room temperature for 24 hours in dark. If the 70% formic acid without cyanobromide was used, the hydrolysis was carried out with gently agitating at room temperature for 24 hours in dark and the reaction mixture was evaporated to dry up by rotary evaporator.

The dried powder was dissolved in 6 mol/L urea and 10 mmol/L Tris solution to form a solution of a pH value more than 7.0. The solution was passed through Ni-NTA resin column again to remove MFH fusion carrier and undigested fusion protein. Pooled elute containing ABP-LK-GLP fusion polypeptide was desalted by use of C18 Sep-Pak column and lyophilized. Finally the recombinant fusion polypeptide was purified using HPLC C18 reverse phase column and eluted with water-acetonitrile gradient solution containing 0.2% formic acid.

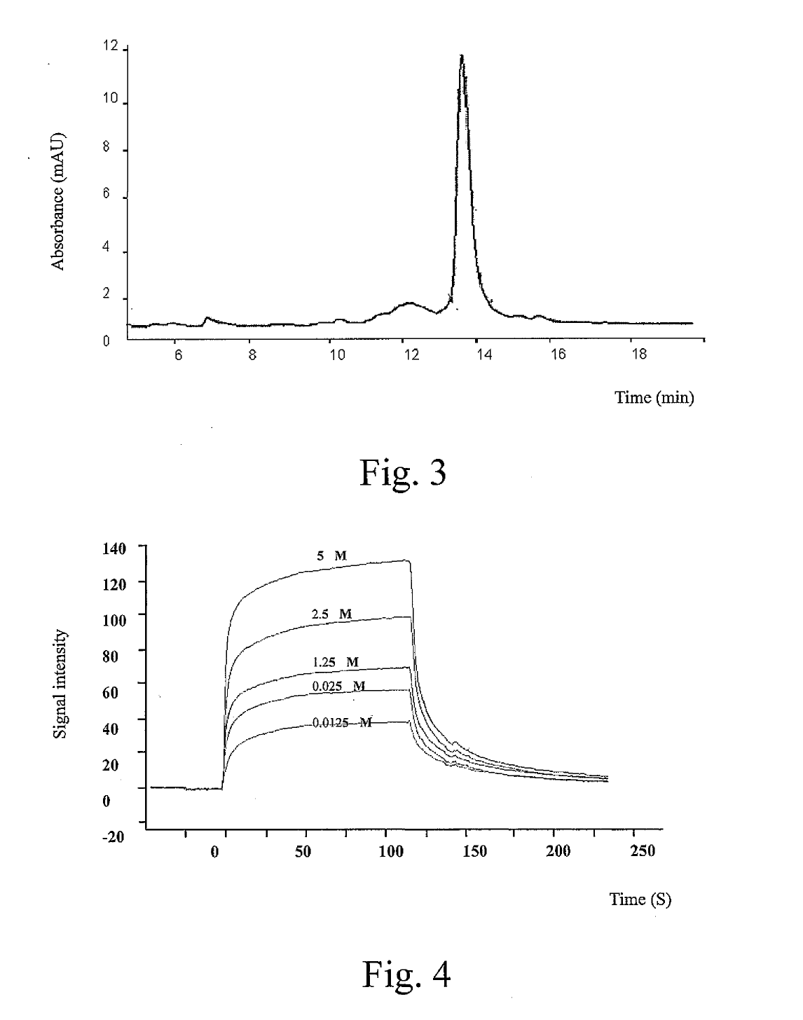

Purified fusion polypeptides were verified by SDS-PAGE, HPLC and mass spectrometry. As shown in FIG. 3, the purity of purified fusion polypeptide was over 99%. Protein concentration was calculated by absorbance at a wavelength of 280 nm (OD₂₈₀ₙₘ) (Gill and von Hipple, Anal Biochem., (1989), 182: 319-26).

### Example 4

### Preparing ABP-LK-PEP Fusion Polypeptide by Solid Phase Method

ABP-LK-PEP fusion polypeptide could also be prepared by amino protection of dimethyl hexehydropyridine carboxamide, conjugation to 2-(2-pyridinone-1-yl)-1,1,3,3-tetramethyl uronium hexafluorophophate, generation of 9-fluoromethoxycarboxy derivatives in a PerSeptive Biosystem peptide synthesizer, and passing through polyethylene-ethylene glycol-polystyrene resins. All fluoromethoxycarboxy amino acid, other chemicals and solvent were commercial available. Analogues of C-terminal amides were prepared with 50 μmol Rink AM resins. The reaction for cleavage of amino acids and deprotection was carried out in a solution containing 90% (v/v) trifluoroacetic acid, 5% (v/v) thioanisole, 3% (v/v) methoxybenzene and 2% (v/v) ethanedithiol to obtain a crude fusion polypeptide. The crude fusion polypeptide was subject to purification by HPLC C18 reverse phase column, wherein the elution solution was water-acetonitrile solution containing 0.1% (v/v) formic acid. Purified fusion polypeptide was verified by mass spectrometry. The purity of each fusion polypeptide was over 99%.

### Example 5

### Amidation of ABP-LK-GLP Fusion Polypeptide

ABP-LK-PEP fusion polypeptide having C-terminal amino group could be prepared by transferring a carboxyl group to the C-terminal of ABP-LK-PEP by enzymology and then removing protecting group by photodegradation.

Amidation was carried out in 50 mmol/L HEPES buffer (pH7.5, containing 5 mmol/L EDTA) or 50 mmol/L CHES buffer (pH9.5, containing mmol/L EDTA). Peptide substrate was dissolved in 5% (v/v) acetic acid to obtain a peptide substrate solution at a concentration of 40 mmol/L. Nucleophilic agent (such as leucine) was dissolved in 50 mmol/L HEPES buffer (pH7.5, containing 5 mmol/L EDTA) to obtain a nuclearphilic agent solution at a concentration of 500 mmol/L. Each 20 μL of peptide subtract solution was mixed with 950 μL nuclearphilic agent solution, followed by being added with carboxylpeptidase at an amount of 25 μL/mL (final concentration of carboxylpeptidase is 0.002 to 0.07 mg/mL). The process was monitored by HPLC. Once there was no product generated, 2.5% (v/v) trifluoroacetic acid was added in to quench the reaction.

Amidation could also be carried out in an organic solvent. A suitable organic solvent includes dimethyl sulfoxide, N,N′-dimethylacetamide, dimethylformamide and the like. The method was as described in Bongers et al., Int. J. Peptide Protein Res., (1992), 40: 268.

Amidation could also be carried out in aqueous solvent. Peptide substrate was dissolved in 5% (v/v) acetic acid to obtain a peptide substrate solution at a concentration of 40 mmol/L. Nuclearphilic agent (such as leucine) was dissolved in 50 mmol/L HEPES buffer (pH7.5, containing 5 mmol/L EDTA) to obtain a nuclearphilic agent solution at a concentration of 500 mmol/L. A 20 μL peptide subtract solution was mixed with 950 μL nuclearphilic agent solution, followed by being added with carboxylpeptidase at an amount of 25 μL/mL to allow final concentration of carboxylpeptidase to be 0.002 to 0.07 mg/mL. The product of the transamidation was ABP-LK-GLP-ONPGA, where ONPGA is O-Nitrophenylglycinamide. The process was monitored by HPLC. Once there was no more product generated, 2.5% (v/v) trifluoroacetic acid was added in to quench the reaction.

The transamidation product, that is ABP-LK-GLP-ONPGA, was subject to cleavage by photodegredation: dissolving ONPGA into 12.5 mL methanol, adding 12.5 mL of 80 mmol/L NaHSO₃ and adjusting pH to 9.5 with 5 mol/L NaOH. The reaction mixture was degassed with N₂ for 15 mins. The photodegradation was carried out by SP200 UV light under nitrogen condition and sampled at 0, 30, 60 and 120 minutes for HPLC analysis. The results were compared with control sample.

### Example 6

### Determination of Affinity of Peptides to Human Serum Albumin by Surface Plasmon Resonance (SPR)

The affinity of fusion polypeptide ABP-LK-GLP to human serum albumin was analyzed with BIAcore 3000 SPR. Human serum albumin was conjugated to CM5 biochip (5000 units), fusion polypeptide ABP-LK-GLP was injected at a concentration of 0, 0.315, 0.625, 1.25, 2.5 and 5 μM and a flow rate of 30 μL/min. The chip was regenerated with 10 mM NaOH, whereby bound peptides could dissociate within 5 min. Signal of conjugated channel subtracted by that of unconjugated channel of injected solution was calculated as the amount of bound peptides during a defined period, as shown in FIG. 4. PBS buffer containing 0.05% Tween-20 was used for dilution of all samples. SPR curve was evaluated by BIAcore™ kinetics evaluation software (Version 4.1). Simulation of 1 to 1 binding model was performed to obtain binding rate (kₒₙ) and dissociating rate (k_{off}). K_{d} was calculated from binding rate (kₒₙ) and dissociating rate (k_{off}), i.e. dissociation constant. The results were summarized in Table 5.

TABLE 5

The dissociation constant (Kd) between ABP-LK-GLP
fusion polypeptide and human serum albumin

| Fusion | The amino acid sequence of | |
|---|---|---|
| polypeptide | ABP included | Kd (M) |
| DP3.1 | DICLPRWGCLWE | 1.03 × 10⁻⁶ |
| DP5.1 | LPWHLKYREPPR | 4.60 × 10⁻⁶ |
| DP6.2 | LPHSHRAHSLPP | 1.44 × 10⁻⁶ |
| DP8.3 | SLLHWTHKIPAL | 2.92 × 10⁻⁶ |

### Example 7

### Sustained Release of LK-GLP-1 by Cleavage of ABP-LK-GLP-1 with Exogenous Thrombin

ABP-LK-GLP-1 fusion polypeptide and human serum albumin were respectively dissolved into 200 μL PBS buffer (pH7.4). The final concentration of ABP-LK-GLP fusion polypeptide was 10 μmol/L and of human serum albumin was 0.3 μmol/L. Human thrombin (0.009 unit) was added into the mixture, followed by being divided into 6 aliquots and hydrolyzed at 37° C. at dark for 24 hours. Samples were respectively obtained at 0, 2, 6, 12, 16 and 24 hours. To each of samples was added trifluoroacetic acid (final concentration of 0.2%) to quench the reaction and samples were then clarified by centrifugation. The supernatant was analyzed by using HPLC and TOF-MASS.

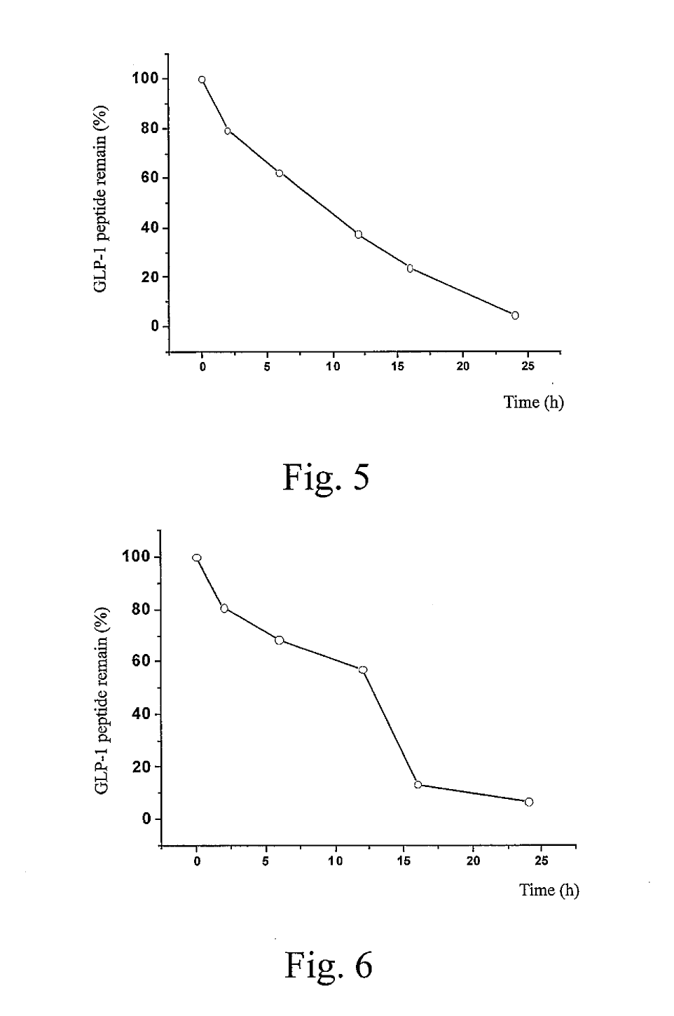

Table 6 listed mass spectrometric (MS) peak area of DP6.2 peptide treated with thrombin for different periods of time. FIG. 5 showed a functional relationship between DP6.2 peptide concentration (i.e. peak area) and time. Thus, the half-life of hydrolysis could be evaluated by FIG. 5 to be about 9 hours.

TABLE 6
Time slope table of single enzyme digestion experiment of
DP6.2 fusion polypeptide by thrombin

| | Time (h) | | | | | |
|---|---|---|---|---|---|---|
| | 0 | 2 | 6 | 12 | 16 | 24 |
| DP6.2 | 10055375.5 | 7974113.2 | 6263427.6 | 3764023.7 | 2394306.8 | 456948.1 |
| peptide MS | | | | | | |
| integral area | | | | | | |
| Area ratio | 100% | 79.3% | 62.3% | 37.4% | 23.8% | 4.5% |

### Example 8

### Sustained Release of Bioactive GLP-1 Peptide a by Cleavage of ABP-LK-GLP-1 with Exogenous Thrombin and DPP IV

ABP-LK-GLP-1 fusion polypeptide and human serum albumin were respectively dissolved into 200 μL PBS buffer (pH7.4). The final concentration of ABP-LK-GLP fusion polypeptide was 10 μM and of human serum albumin was 0.3 μM. Human thrombin (0.009 unit) and DPP IV (0.0016 ng) were added into the mixture, followed by being divided into 6 aliquots and hydrolyzed at 37° C. at dark for 24 hours. Samples were respectively obtained at 0, 2, 6, 12, 16 and 24 hours and each of samples was boiled for two minutes to quench the reaction and centrifuged. The resulting supernatant was analyzed by using HPLC and TOF-mass spectrometry.

Table 7 listed mass spectrometric (MS) peak area of DP3.1 fusion polypeptide treated with enzyme for different periods of time. FIG. 6 showed a functional relationship between peak area of DP3.1 fusion polypeptide hydrolyzed with human thrombin and DPP IV and time. Thus, the half-life of enzyme digestion (i.e. the releasing half-life of active polypeptide) could be evaluated by FIG. 8 to be about 13 hours.

TABLE 7
Time slope table of double enzyme digestion experiment of DP3.1
fusion polypeptide by using thrombin and DPP IV

| | time (h) | | | | | |
|---|---|---|---|---|---|---|
| | 0 | 2 | 6 | 12 | 16 | 24 |
| DP3.1 | 28079570.3 | 22632133.7 | 19234505.7 | 15977275.5 | 3743753.1 | 1881331.2 |
| peptide MS | | | | | | |
| integral | | | | | | |
| area | | | | | | |
| The ratio of | 100% | 80.6 | 68.5% | 56.9% | 13.3% | 6.7% |
| area | | | | | | |

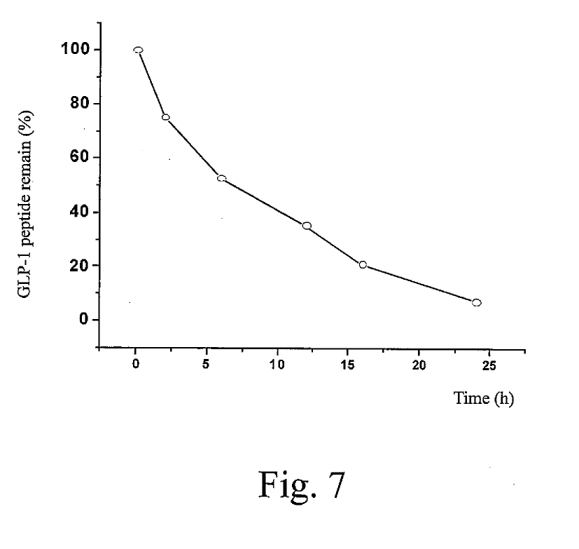

Table 8 listed MS peak area of DP4.1 fusion polypeptide corresponding to cleavage by thrombin and DPP IV for different periods of time. FIG. 7 showed a functional relationship between peak area of DP4.1 fusion polypeptide hydrolyzed with human thrombin and DPP IV and time. Thus, the half-life of enzyme digestion (i.e. the releasing half-life of active polypeptide) could be evaluated by FIG. 9 to be about 7 hours.

TABLE 8
Time-course experiments of double enzyme digestion of DP4.1 fusion
polypeptide by using thrombin and DPP IV

| | Time (h) | | | | | |
|---|---|---|---|---|---|---|
| | 0 | 2 | 6 | 12 | 16 | 24 |
| DP4.1 | 12626709.1 | 9482015.7 | 6652133.7 | 4459186.4 | 2643753.6 | 912774.9 |
| peptide MS | | | | | | |
| integral | | | | | | |
| area | | | | | | |
| The ratio of | 100% | 75.1% | 52.7% | 35.3% | 20.9% | 7.2% |
| area | | | | | | |

### Example 9

### The Activation of GLP-1 Receptor by GLP-1 Peptide Released from ABP-LK-GLP-1

In Example 8, after being treated with thrombin and DPP IV, GLP-1 was released from ABP-LK-GLP, 50 μmol/L of DDP IV inhibitor (Linco, St. Charles, Mo.) and 50 μmol/L of PPACK were added to quench the reaction. GLP-1 released from ABP-LK-GLP competed with [¹²⁵I] GLP-1 to bind to GLP-1 receptor while being incubated with CHO/GLP-1R. The actual experimental steps were extracted from method as described by Montrose-Rafizadeh with proper modification as following:

(1) Transforming CHO cells with GLP-1 receptor plasmid and culturing in 12-well plate, washing with Ham's F12 culture medium free of serum at two hours before the experiment, washing with 0.5 mL Ham's F12 culture medium twice, then culturing in Ham's F12 culture medium containing 2% (w/v) BSA and 10 mmol/L. glucose at 4° C. overnight and adding sustained release GLP-1 peptide and 30,000 cpm ¹²⁵I-GLP-1 (GE Life Science, QC) to the culture medium.

(2) After cultured, removing supernatant, washing cells with cold PBS for three times, and then mixing with 0.5 mL 0.5 mol/L NaOH and 0.1% (mg/mL) SDS for 10 minutes. Irradiation of the cell lysis was determined by Apec-Series λ-counter (ICN Biomedicals, Inc., Costa Mesa, Calif.). The values of IC₅₀ were listed in Table 9.

TABLE 9

Analysis of receptor binding affinities of ABP-LK-GLP fusion polypeptide
and there effects on the production of cAMP in CHO/GLP-1R system

| | | Receptor | cAMP |
|---|---|---|---|
| | | binding affinity | production |
| Amino acid sequence | name | IC₅₀ (nM) | EC₅₀ (nM) |
| GLP-1(7-36)-NH₂ | GLP-1(7-37) | 0.3 ± 0.06 | 3.1 ± 0.8 |
| GLP-1(7-37)-OH | GLP-1(7-36) | 2.4 ± 0.6 | 20 ± 2.1 |
| FNPRHAEGTFTSDVSSYLEGQAAKEFIAWLVKG | FN-GLP | 450 ± 13 | >1000 |
| RG | | | |
| LVPRHAEGTFTSDVSSYLEGQAAKEFIAWLVKG | LV-GLP | 467 ± 14 | >1000 |
| RG | | | |
| PDICLPRWGCLWEFNPRGA | DP3.1 | 2.1 ± 0.5 | 23 ± 2.0 |
| HAEGTFTSDVSSYLEGQAAKEFIAWLVKGRG | | | |
| PDICLPRWGCLWEFNPRGP | DP3.2 | 2.3 ± 0.7 | 19 ± 1.5 |
| HAEGTFTSDVSSYLEGQAAKEFIAWLVKGR | | | |
| G | | | |
| PDICLPRWGCLWEFNPRGS | DP3.3 | 2.3 ± 0.5 | 21 ± 2.1 |
| HAEGTFTSDVSSYLEGQAAKEFIAWLVKGRG | | | |
| PNICLPRWGCLWDFNPRGA | DP4.1 | 2.5 ± 0.8 | 19 ± 2.1 |
| HAEGTFTSDVSSYLEGQAAKEFIAWLVKGRG | | | |
| PNICLPRWGCLWDFNPRGP | DP4.2 | 2.4 ± 0.7 | 22 ± 2.2 |
| HAEGTFTSDVSSYLEGQAAKEFIAWLVKGRG | | | |
| PNICLPRWGCLWDFNPRGS | DP4.3 | 2.1 ± 0.7 | 18 ± 2.2 |
| HAEGTFTSDVSSYLEGQAAKEFIAWLVKGRG | | | |
| PLPHSHRAHSLPPFNPRGA | DP6.1 | 2.0 ± 0.7 | 23 ± 2.2 |
| HAEGTFTSDVSSYLEGQAAKEFIAWLVKGRG | | | |
| PLPHSHRAHSLPPFNPRGP | DP6.2 | 2.1 ± 0.8 | 23 ± 2.1 |
| HAEGTFTSDVSSYLEGQAAKEFIAWLVKGRG | | | |
| PLPHSHRAHSLPPFNPRGS | DP6.3 | 2.2 ± 0.6 | 20 ± 2.1 |
| HAEGTFTSDVSSYLEGQAAKEFIAWLVKGRG | | | |
| PSLLHWTHKIPALFNPRGA | DP8.1 | 2.2 ± 0.6 | 24 ± 2.4 |
| HAEGTFTSDVSSYLEGQAAKEFIAWLVKGRG | | | |
| PSLLHWTHKIPALFNPRGP | DP8.2 | 1.9 ± 0.5 | 22 ± 2.2 |
| HAEGTFTSDVSSYLEGQAAKEFIAWLVKGRG | | | |
| PSSLLHWTHKIPALFNPRGS | DP8.3 | 2.5 ± 0.6 | 23 ± 2.2 |
| HAEGTFTSDVSSYLEGQAAKEFIAWLVKGRG | | | |
| DICLPRWGCLWEFNPRGA | M3.1 | 2.2 ± 0.5 | 22 ± 2.0 |
| HAEGTFTSDVSSYLEGQAAKEFIAWLVKGRG | | | |
| DICLPRWGCLWEFNPRGP | M3.2 | 2.1 ± 0.7 | 21 ± 2.1 |
| HAEGTFTSDVSSYLEGQAAKEFIAWLVKGRG | | | |
| DICLPRWGGLWEFNPRGS | M3.3 | 2.4 ± 0.5 | 22 ± 2.1 |
| HAEGTFTSDVSSYLEGQAAKIEFIAWLVKGRG | | | |
| NICLPRWGCLWDFNPRGA | M4.1 | 2.4 ± 0.8 | 21 ± 2.1 |
| HAEGTFTSDVSSYLEGQAAKEFIAWLVKGRG | | | |
| NICLPRWGCLWDFNPRGP | M4.2 | 2.2 ± 0.7 | 21 ± 2.2 |
| HAEGTFTSDVSSYLEGQAAKEFIAWLVKGRG | | | |
| NICLPRWGCLWDFNPRGS | M4.3 | 2.2 ± 0.7 | 19 ± 2.2 |
| HAEGTFTSDVSSYLEGQAAKEFIAWLVKGRG | | | |
| LPHSHRAHSLPPFNPRGA | M6.1 | 2.2 ± 0.7 | 24 ± 2.2 |
| HAEGTFTSDVSSYLEGQAAKEFIAWLVKGRG | | | |
| LPHSHRAHSLPPFNPRGP | M6.2 | 2.3 ± 0.8 | 21 ± 2.1 |
| HAEGTFTSDVSSYLEGQAAKEFIAWLVKGRG | | | |
| LPHSHRAHSLPPFNPRGS | M6.3 | 2.4 ± 0.6 | 23 ± 2.1 |
| HAEGTFTSDVSSYLEGQAAKEFIAWLVKGRG | | | |
| SLLHWTHKIPALFNPRGA | M8.1 | 2.2 ± 0.6 | 24 ± 2.4 |
| HAEGTFTSDVSSYLEGQAAKEFIAWLVKGRG | | | |
| SLLHWTHKIPALFNPRGP | M8.2 | 1.9 ± 0.5 | 21 ± 2.2 |
| HAEGTFTSDVSSYLEGQAAKEFIAWLVKGRG | | | |
| SLLHWTHKIPALFNPRGS | M8.3 | 2.2 ± 0.6 | 21 ± 2.2 |
| HAEGTFTSDVSSYLEGQAAKEFIAWLVKGRG | | | |
| LPHSHRAHSLPPFNPRPP | PP6.2 | 2.3 ± 0.8 | 21 ± 2.1 |
| HAEGTFTSDVSSYLEGQAAKEFIAWLVKGRG | | | |
| LPHSHRAHSLPPFNPRPA | PA6.2 | 2.2 ± 0.7 | 23 ± 2.4 |
| HAEGTFTSDVSSYLEGQAAKEFIAWLVKGRG | | | |

### Example 10

### The Effect of GLP-1 Released from ABP-LK-GLP on Generation of cAMP in Cells

CHO cells transformed with GLP-1 were cultured in 12-well plates to a cell density of 60% to 70%, followed by washing with Krebs-Ringer phosphate buffer for three times. Before examination, 1 mL KRP solution (containing 0.1% BSA and 1 mmg/mL IBMX) was added, followed by adding GLP-1 for analysis for 30 minutes and then washing cells with cold PBS for three times to quench the reaction. In control experiment, no GLP-1 was added. Samples were treated with 1 mL cold perchloric acid (0.6 mg/mL) for 5 minutes to release cellular cAMP. pH value of the samples was adjusted to 7 with 84 μL of mg/mL potassium carbonate. Samples were rotated and then centrifuged for 5 minutes (2000 g, 4° C.) to acquire precipitation. Supernatant was removed by vacuum and then the precipitation was dissolved into 300 μL of 0.05 mg/mL Tris buffer (pH7.5, containing 4 mmg/mL EDTA), added with sodium bicarbonate (0.15 mg/mL) and zinc sulfate (0.15 mg/mL), followed by being placed on ice for 15 minutes. Centrifugation was performed at 2000 g at 4° C. for minutes to remove precipitation and obtain a supernatant. The supernatant was analyzed with [³H] cAMP competition assay kit (Amersham Biotech, QC). The values of EC₅₀ were listed in Table 9.

Table 9 showed that GLP-1 released from ABP-LK-GLP had high biological activity.

### Example 11

### Determination of the Half-Life of ABP-LK-GLP Fusion Polypeptides in Mouse Model using GLP-1 Antibody

The present example employed competitive enzyme-linked immunoassay using biotin-conjugated antibody with specific affinity to GLP-1(7-36) to determine the amount of GLP-1 released from ABP-LK-GLP in human plasma sample. 96-well plate was coated with goat anti-mouse IgG antibody. GLP-1 standard or samples, labeled antigen and GLP-1 antibody were added into each well for competitive immunoassay. After culture plate were mixed and washed, streptoavidin labeled with HRP(SA-HRP) were added onto wells to form a complex of HRP-conjugated streptoavidin-biotin-GLP-1 antibody. Finally, the activity of HRPase was determined by o-phenylenediamine (OPD) and the concentration of GLP-1 was calculated.

Before the test began, all reagents were deposited under room temperature. Each well of multiwell microplates (UltiDent, QC) was coated with 50 μL of 40 μg/mL ovalbumin and the microplates were placed at 4° C. overnight. The multiwell microplates were washed with 0.35 mL/well PBS containing 0.1% Tween-20 twice, followed by being placed at 37° C. and blocked with BSA for 1 hour. Mouse serum was added to a final volume of 100 μL for washing the plate. To each well was added 4 μl labeled antigen solution first, followed by being added with the sample and 4 μL of GLP-1 antibody. In control experiments, to each well was added 3 μL standard solution to form a concentration gradient of 0, 0.206, 0.617, 1.852, 5.556, 16.67, 50 ng/mL. The plate was sealed with parafilm, maintained at 4° C. for 16 to 18 hours and washed with. To each well 10 μL of SA-HRP solution was added. The plate was covered by parafilm, agitated for 1 hour at room temperature on culture plate agitator and washed with 0.35 mL/well PBS solution for five times. To each well was added 10 μL substrate solution and maintained at room temperature for 30 minutes. 10 μL of SA-stop solution was added to each well to quench the reaction. Light absorbance was examined at 492 nm. Absorbance of standard wells were calculated and plotted to obtain a standard curve. GLP-1 concentrations of samples were determined by absorbance value in the corresponding standard curve. The results were listed in Table 10.

TABLE 10

Analysis of the half-life of ABP-LK-GLP-1
fusion polypeptide in mouse model

| | | | | |
|---|---|---|---|---|
| | | Half-life | Half-life | Half-life |
| | | t_{1/2} | t_{1/2} | t_{1/2} |
| | | (Hours) | (Hours) | (Hours) |
| | | 11 μg | 100 μg | 150 μg |
| Name | Amino acid sequence | polypeptide | polypeptide | polypeptide |

| | | | | |
|---|---|---|---|---|
| GLP-1(7-37) | GLP-1(7-36)-NH₂ | 0.031 | 0.032 | 0.032 |
| GLP-1(7-36) | GLP-1(7-37)-OH | 0.032 | 0.031 | 0.032 |
| FN-GLP | FNPRHAEGTFTSDVSSYLEGQAAKEFIAWLV | 2.3 | 2.4 | 3.1 |
| | KGRG | | | |
| DP6.2 | PLPHSHRAHSLPPFNPRGP | 5.3 | 5.6 | 12.5 |
| | HAEGTFTSDVSSYLEGQAAKEFIAWLVKGRG | | | |
| PP6.2 | LPHSHRAHSLPPFNPRPP | 35.6 | 49.5 | 98.3 |
| | HAEGTFTSDVSSYLEGQAAKEFIAWLVKGRG | | | |
| PA6.2 | LPHSHRAHSLPPFNPRPA | 26.4 | 32.7 | 65.4 |
| | HAEGTFTSDVSSYLEGQAAKEFIAWLVKGRG | | | |

### Example 12

### Analysis of the Half-Life of ABP-LK-CT Fusion Polypeptides in Mouse Model

Calcitonin (CT) is a polypeptide hormone consisting of 32 amino acids. Since Calcitonin was found by Coop et al., physiologists in Canada, people widely investigated on its structure, physiology and pharmacology and consecutively brought breakthrough. The clinical application of calcitonin expanded and become more and more important with the progress of the thorough research. Calcitonin inhibited either the osseous absorption or osteolysis to reduce release of calcium from bones, meanwhile bones absorbed calcium in plasma to lead to decrease blood calcium. Calcitonin could also inhibit dissolving and transferring of bone mineral, inhibit bone matrix degradation, enhance bone regeneration, increase excretion of urinary calcium and urinary phosphorus, and induce hypocalcemia and hypophosphatemia. Calcitonin acted to decrease blood calcium within only a short period in physics and could counteract the effect of parathyroid hormone on bones.

Calcitonin and its fusion polypeptide ABP-LK-CT according to the present invention were prepared by the method according to Example 3 and Example 5. Analysis of the half-life of ABP-LK-CT fusion polypeptide in mouse model was performed by a method similar to the method described in Example 12 except for using calcitonin antibody. The results were shown in Table 11. Fusion polypeptide could enhance the half-life of calcitonin in blood up to 70 times.

TABLE 11

Analysis of the half-life of ABP-LK-CT fusion
polypeptide in mouse model

| | | | |
|---|---|---|---|
| | | Half-life | Half-life |
| | | t_{1/2} | t_{1/2} |
| | | (hours) | (hours) |
| | | 11 μg | 200 μg |
| Amino acid sequence | name | polypeptide | polypeptide |

| | | | |
|---|---|---|---|
| CSNLSTCVLGKLSQELHKLQT | CT | 1.25 | 2.11 |
| YPRTNTGSGTP-NH₂ | | | |
| LPHSHRAHSLPPFNPRGPCSN | ABP- | 37.8 | 76.3 |
| LSTCVLGKLSQELHKLQTYPR | LK-CT | | |
| TNTGSGTP-NH₂ | | | |

### Example 13

### Analysis of the Half-Life of Bcl-2 Family Protein Binding Peptide and ABP Constructed Fusion Polypeptide in Mouse Model

Bax is an apoptotic protein. The synthetic peptide of its BH3 binding domain could induce cell apoptosis. When mutation occurred in BH3 of Bax, Bax would loss its binding ability to BCl-X_{L}, which resulted in that the mutated Bax was unable to induce cell apoptosis. A truncated polypeptide derived from BH3 domain consisting of 14 amino acids (58-71), i.e. RYGRELRRMSDEFE, showed a binding activity to Bcl-X_{L} and could induce apoptosis. Synthetic BH3 peptides were shown to have effects on several cancer cells.

BH3 peptide and its fusion polypeptide ABP-LK-BH3 according to the present invention were prepared by the method as described in Example 4. The half-life of ABP-LK-BH3 fusion polypeptide in mouse model was analyzed by a method similar to Example 12 with BH3 antibody. The results were listed in Table 12. Fusion polypeptide can enhance the half-life of BH3 in blood up to 195 times.

TABLE 12

Analysis of the half-life of ABP-LK-BH3 fusion
polypeptide comprising Bc1-2 family protein
binding peptide in mouse model

| | | | |
|---|---|---|---|
| | | Half-life | Half-life |
| | | t_{1/2} | t_{1/2} |
| | | (hours) | (hours) |
| | | 12 μg | 100 μg |
| Amino acid sequence | Name | polypeptide | polypeptide |

| | | | |
|---|---|---|---|
| RYGRELRRMSDEFE | BH3 | 0.4 | 0.74 |
| LPHSHRAHSLPPFNPRGA | ABP-LK- | 42.3 | 86.5 |
| RYGRELRRMSDEFE | BH3 | | |

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A method for sustainedly releasing bioactive peptide from a fusion polypeptide, comprising
providing a fusion polypeptide including a bioactive peptide conjugated to a serum albumin binding peptide through a cleavable molecular linker, wherein the molecular linker is sensitive to plasma environment and serves as a switch to sustainedly release bioactive peptide therein; and
administrating a host the fusion polypeptide, whereby plasma proteinases or mild alkalinity of blood in the host can catalytically cleave the molecular linker to release the bioactive peptide therein.

2. The method of claim 1, wherein the serum albumin binding peptide has an amino acid sequence as following formula (I):
(I)
Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂,
wherein
Xaa₁ is leucine;
Xaa₂ is proline;
Xaa₃ is any amino acid except cysteine;
Xaa₄ is any amino acid;
Xaa₅ is any amino acid;
Xaa₆ is a positively charged amino acid;
Xaa₇ is a hydrophobic amino acid;
Xaa₈ is a positively charged amino acid;
Xaa₉ is any amino acid except cysteine;
Xaa₁₀ is a hydrophobic amino acid;
Xaa₁₁ is proline; and
Xaa₁₂ is any amino acid.

3. The method of claim 2, wherein the serum albumin binding peptide is Leu-Pro-Trp-His-Leu-Lys-Tyr-Arg-Glu-Pro-Pro-Arg or Leu-Pro-His-Ser-His-Arg-Ala-His-Ser-Leu-Pro-Pro.

4. The method of claim 1, wherein the molecular linker has an amino acid sequence of thrombin recognition site, wherein said amino acid sequence comprises an amino acid sequence of the following formula (II):

| | |
|---|---|
| Xaaⱼ-Xaaₖ-Xaaᵢ-Arg-Xaaₘ-Xaaₙ, | (II) |

wherein
Xaaⱼ is a hydrophobic amino acid or peptidyl bond;
Xaaₖ is a hydrophobic amino acid or peptidyl bond;
Xaaᵢ is proline or valine;
Xaaₘ is a non-acidic amino acid or peptidyl bond; and
Xaaₙ is a non-acidic amino acid or peptidyl bond.

5. The method of claim 4, wherein the molecular linker is Phe-Asn-Pro-Arg-Gly-Ala, Phe-Asn-Pro-Arg-Gly-Ser, Phe-Asn-Pro-Arg-Gly-Pro, Phe-Asn-Pro-Arg-Pro-Pro or Phe-Asn-Pro-Arg-Pro-Ala.

6. The method of claim 1, wherein the molecular linker is a disulfide bond.

7. The method of claim 1, wherein the serum albumin binding peptide is Ser-Leu-Phe-Arg-His-Gln-His-Ala-Thr-Pro-Gln-Ile, Ser-Leu-Leu-His-Trp-Thr-His-Lys-Ile-Pro-Ala-Leu or Lys-Tyr-Asn-His-Ser-Hlis-Lys-Tyr-Trp-Gln-Arg-Pro.

8. The method of claim 1, wherein the bioactive peptide is human glucagon-like peptide-1, calcitonin or any one of peptides binding to Bcl-2 family apoptotic proteins.

9. The method of claim 8, wherein the peptide binding to Bcl-2 family apoptotic protein is a BH3 peptide derived from Bax protein.

10. A method for using the fusion polypeptide according to claim 1 to treat human type 2 diabetes.

11. A method for using the fusion polypeptide according to claim 1 to treat human osteoporosis.

12. A method for using the fusion polypeptide according to claim 1 to treat human cancer.


SEQUENCE LISTING (44 sequences)

SEQ ID NO: 1 - protein, 12 aa, Artificial
  peptide with high affinity to human serum albumin
NVCLPKWGCLWE

SEQ ID NO: 2 - protein, 12 aa, Artificial
  peptide with high affinity to human serum albumin
DVCLPQWGCLWG

SEQ ID NO: 3 - protein, 12 aa, Artificial
  peptide with high affinity to human serum albumin
DICLPRWGCLWE

SEQ ID NO: 4 - protein, 12 aa, Artificial
  peptide with high affinity to human serum albumin
NICLPRWGCLWD

SEQ ID NO: 5 - protein, 12 aa, Artificial
  peptide with high affinity to human serum albumin
LPWHLKYREPPR

SEQ ID NO: 6 - protein, 12 aa, Artificial
  peptide with high affinity to human serum albumin
LPHSHRAHSLPP

SEQ ID NO: 7 - protein, 12 aa, Artificial
  peptide with high affinity to human serum albumin
SLFRHQHATPQI

SEQ ID NO: 8 - protein, 12 aa, Artificial
  peptide with high affinity to human serum albumin
SLLHWTHKIPAL

SEQ ID NO: 9 - protein, 12 aa, Artificial
  peptide with high affinity to human serum albumin
KYNHSHLYWQRP

SEQ ID NO: 10 - protein, 30 aa, Artificial
  native structure generated from human GLP-1, GLP (7-36)
HAEGTFTSDVSSYLEGQAAKEFIAWLVKGR

SEQ ID NO: 11 - protein, 31 aa, Artificial
  native structure generated from human GLP-1, GLP (7-37)
HAEGTFTSDVSSYLEGQAAKEFIAWLVKGRG

SEQ ID NO: 12 - protein, 50 aa, Artificial
  sustained release human glucagon-like peptide-1 analogue, DP3.1
PDICLPRWGCLWEFNPRGAHAEGTFTSDVSSYLEGQAAKEFIAWLVKGRG

SEQ ID NO: 13 - protein, 50 aa, Artificial
  sustained release human glucagon-like peptide-1 analogue, DP3.2
PDICLPRWGCLWEFNPRGPHAEGTFTSDVSSYLEGQAAKEFIAWLVKGRG

SEQ ID NO: 14 - protein, 50 aa, Artificial
  sustained release human glucagon-like peptide-1 analogue, DP3.3
PDICLPRWGCLWEFNPRGSHAEGTFTSDVSSYLEGQAAKEFIAWLVKGRG

SEQ ID NO: 15 - protein, 50 aa, Artificial
  sustained release human glucagon-like peptide-1 analogue, DP4.1
PNICLPRWGCLWDFNPRGAHAEGTFTSDVSSYLEGQAAKEFIAWLVKGRG

SEQ ID NO: 16 - protein, 50 aa, Artificial
  sustained release human glucagon-like peptide-1 analogue, DP4.2
PNICLPRWGCLWDFNPRGPHAEGTFTSDVSSYLEGQAAKEFIAWLVKGRG

SEQ ID NO: 17 - protein, 50 aa, Artificial
  sustained release human glucagon-like peptide-1 analogue, DP4.3
PNICLPRWGCLWDFNPRGSHAEGTFTSDVSSYLEGQAAKEFIAWLVKGRG

SEQ ID NO: 18 - protein, 50 aa, Artificial
  sustained release human glucagon-like peptide-1 analogue, DP6.1
PLPHSHRAHSLPPFNPRGAHAEGTFTSDVSSYLEGQAAKEFIAWLVKGRG

SEQ ID NO: 19 - protein, 50 aa, Artificial
  sustained release human glucagon-like peptide-1 analogue, DP6.2
PLPHSHRAHSLPPFNPRGPHAEGTFTSDVSSYLEGQAAKEFIAWLVKGRG

SEQ ID NO: 20 - protein, 50 aa, Artificial
  sustained release human glucagon-like peptide-1 analogue, DP6.3
PLPHSHRAHSLPPFNPRGSHAEGTFTSDVSSYLEGQAAKEFIAWLVKGRG

SEQ ID NO: 21 - protein, 50 aa, Artificial
  sustained release human glucagon-like peptide-1 analogue, DP8.1
PSLLHWTHKIPALFNPRGAHAEGTFTSDVSSYLEGQAAKEFIAWLVKGRG

SEQ ID NO: 22 - protein, 50 aa, Artificial
  sustained release human glucagon-like peptide-1 analogue, DP8.2
PSLLHWTHKIPALFNPRGPHAEGTFTSDVSSYLEGQAAKEFIAWLVKGRG

SEQ ID NO: 23 - protein, 51 aa, Artificial
  sustained release human glucagon-like peptide-1 analogue, DP8.3
PSSLLHWTHKIPALFNPRGSHAEGTFTSDVSSYLEGQAAKEFIAWLVKGRG

SEQ ID NO: 24 - protein, 49 aa, Artificial
  sustained release human glucagon-like peptide-1 analogue, M3.1
DICLPRWGCLWEFNPRGAHAEGTFTSDVSSYLEGQAAKEFIAWLVKGRG

SEQ ID NO: 25 - protein, 49 aa, Artificial
  sustained release human glucagon-like peptide-1 analogue, M3.2
DICLPRWGCLWEFNPRGPHAEGTFTSDVSSYLEGQAAKEFIAWLVKGRG

SEQ ID NO: 26 - protein, 49 aa, Artificial
  sustained release human glucagon-like peptide-1 analogue, M3.3
DICLPRWGCLWEFNPRGSHAEGTFTSDVSSYLEGQAAKEFIAWLVKGRG

SEQ ID NO: 27 - protein, 49 aa, Artificial
  sustained release human glucagon-like peptide-1 analogue, M4.1
NICLPRWGCLWDFNPRGAHAEGTFTSDVSSYLEGQAAKEFIAWLVKGRG

SEQ ID NO: 28 - protein, 49 aa, Artificial
  sustained release human glucagon-like peptide-1 analogue, M4.2
NICLPRWGCLWDFNPRGPHAEGTFTSDVSSYLEGQAAKEFIAWLVKGRG

SEQ ID NO: 29 - protein, 49 aa, Artificial
  sustained release human glucagon-like peptide-1 analogue, M4.3
NICLPRWGCLWDFNPRGSHAEGTFTSDVSSYLEGQAAKEFIAWLVKGRG

SEQ ID NO: 30 - protein, 49 aa, Artificial
  sustained release human glucagon-like peptide-1 analogue, M6.1
LPHSHRAHSLPPFNPRGAHAEGTFTSDVSSYLEGQAAKEFIAWLVKGRG

SEQ ID NO: 31 - protein, 49 aa, Artificial
  sustained release human glucagon-like peptide-1 analogue, M6.2
LPHSHRAHSLPPFNPRGPHAEGTFTSDVSSYLEGQAAKEFIAWLVKGRG

SEQ ID NO: 32 - protein, 49 aa, Artificial
  sustained release human glucagon-like peptide-1 analogue, M6.3
LPHSHRAHSLPPFNPRGSHAEGTFTSDVSSYLEGQAAKEFIAWLVKGRG

SEQ ID NO: 33 - protein, 49 aa, Artificial
  sustained release human glucagon-like peptide-1 analogue, M8.1
SLLHWTHKIPALFNPRGAHAEGTFTSDVSSYLEGQAAKEFIAWLVKGRG

SEQ ID NO: 34 - protein, 49 aa, Artificial
  sustained release human glucagon-like peptide-1 analogue, M8.2
SLLHWTHKIPALFNPRGPHAEGTFTSDVSSYLEGQAAKEFIAWLVKGRG

SEQ ID NO: 35 - protein, 49 aa, Artificial
  sustained release human glucagon-like peptide-1 analogue, M8.3
SLLHWTHKIPALFNPRGSHAEGTFTSDVSSYLEGQAAKEFIAWLVKGRG

SEQ ID NO: 36 - protein, 49 aa, Artificial
  sustained release human glucagon-like peptide-1 analogue, PP6.2
LPHSHRAHSLPPFNPRPPHAEGTFTSDVSSYLEGQAAKEFIAWLVKGRG

SEQ ID NO: 37 - protein, 49 aa, Artificial
  sustained release human glucagon-like peptide-1 analogue, PA6.2
LPHSHRAHSLPPFNPRPAHAEGTFTSDVSSYLEGQAAKEFIAWLVKGRG

SEQ ID NO: 38 - protein, 50 aa, Artificial
  DP5.1
PLPWHLKYREPPRFNPRGAHAEGTFTSDVSSYLEGQAAKEFIAWLVKGRG

SEQ ID NO: 39 - protein, 35 aa, Artificial
  FN-GLP
FNPRHAEGTFTSDVSSYLEGQAAKEFIAWLVKGRG

SEQ ID NO: 40 - protein, 35 aa, Artificial
  LV-GLP
LVPRHAEGTFTSDVSSYLEGQAAKEFIAWLVKGRG

SEQ ID NO: 41 - protein, 32 aa, Artificial
  CT
CSNLSTCVLGKLSQELHKLQTYPRTNTGSGTP

SEQ ID NO: 42 - protein, 50 aa, Artificial
  ABP-LK-CT
LPHSHRAHSLPPFNPRGPCSNLSTCVLGKLSQELHKLQTYPRTNTGSGTP

SEQ ID NO: 43 - protein, 14 aa, Artificial
  BH3
RYGRELRRMSDEFE

SEQ ID NO: 44 - protein, 32 aa, Artificial
  ABP-LK-BH3
LPHSHRAHSLPPFNPRGARYGRELRRMSDEFE